(19) United States
(12) Patent Application Publication
(10) Pub. No.: US 2020/0375806 A1
(43) Pub. Date: Dec. 3, 2020

(54) GERIATRIC UNDERGARMENT PROVIDING AN ARRANGEMENT OF STRUCTURAL PROTECTION AGAINST PRESSURE ULCERS

(71) Applicant: Marco Aurelio Alves de Souza, Orlando, FL (US)

(72) Inventor: Marco Aurelio Alves de Souza, Orlando, FL (US)

(21) Appl. No.: 16/425,025

(22) Filed: May 29, 2019

(51) Int. Cl. A61F 13/15; A61F 5/34

(52) U.S. Cl. CPC A61F 13/15; A61F 2013/49098; A61F 2013/15024; A61F 5/34

(57) ABSTRACT

A geriatric undergarment is provided. The geriatric undergarment dimensioned and adapted to be worn about the waist of a human wearer. The geriatric undergarment may provide coated, pressure absorbent material disposed along an inner surface so as to be disposed along diaper pressure regions of the user, including the right and left lateral, sacral, or posterior and trochanteric and/or posterior dorsal regions. The coated, pressure absorbent material may be shaped in pyramidal or tapered form.

### BACKGROUND OF THE INVENTION

The present invention relates to geriatric undergarments and, more particularly, to a geriatric undergarment providing structural protection against pressure ulcers.

Pressure ulcers, one of the evils caused by laying in the same positioning for a long period of time, is an ailment that affects a vast majority of the bedridden patients, generating a high cost of treatment, suffering and pain for the patient. Furthermore, pressure ulcers may result in serious complications such as generalized infections and even the death.

Disposable diapers are used by bedridden patients with reduced or no mobility, who need help in physiological activities. Such disposable diapers can cause pressure ulcers, where there is more friction, or more pressure, resulting from reduced mobility and bad distributed pressure over the skin.

As can be seen, there is a need for a geriatric undergarment providing an arrangement of structural protection against pressure ulcers. The present invention embodies a diaper that aims to protect body areas of greater contact and friction, such as the trochanters and the sacrum, offering comfort and preventing skin ulceration in the case of long-term pressure against the bed or wheelchair or other circumstances of low mobility common among patients of long-term care facilities.

### SUMMARY OF THE INVENTION

In one aspect of the present invention, a geriatric undergarment includes the following: a body dimensioned and adapted to be worn about the waist of a human wearer; and one or more pressure absorbent attachment points disposed along an inner surface of the body so that each pressure absorbent material is located adjacent to one or more diaper pressure regions of the wearer.

In another aspect of the present invention, the geriatric undergarment includes the following: a body of impact resistant foam dimensioned and adapted to be worn about the waist of a human wearer; and one or more pressure absorbent attachment points adhered via adhesive along an inner surface of the body so that each pressure absorbent material is located adjacent to the one or more diaper pressure regions of the wearer including a lateral, sacral, posterior, trochanteric and a posterior dorsal region, wherein the one or more of the pressure absorbent attachment points are shaped in pyramidal or a tapered form.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention in an open condition;

FIG. 2 is a perspective view of an exemplary embodiment of the present invention in a closed condition;

FIG. 3 is a perspective view of an exemplary embodiment of the present invention in the open condition; and

FIG. 4 is a perspective view of an exemplary embodiment of the present invention in the open condition.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a geriatric undergarment dimensioned and adapted to be worn about the waist of a human wearer. The geriatric undergarment may provide coated, pressure absorbent material disposed along an inner surface so as to be disposed along diaper pressure regions of the user, including the right and left lateral, sacral, or posterior and trochanteric and/or posterior dorsal regions. The coated, pressure absorbent material may be shaped in pyramidal or tapered form.

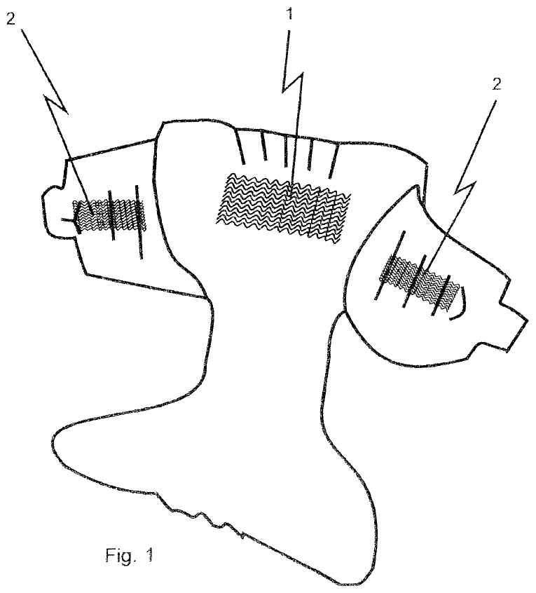

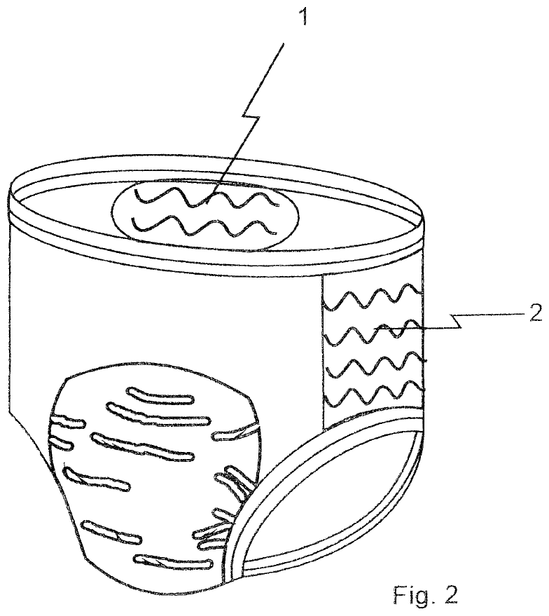

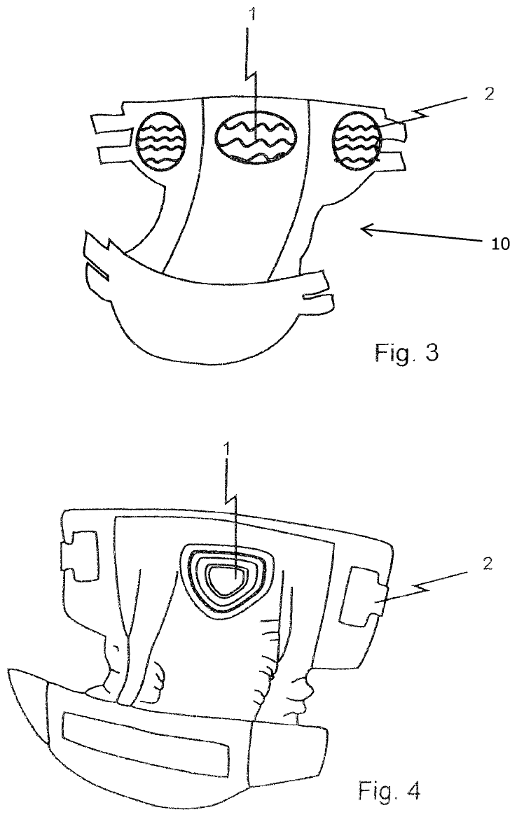

Referring to FIGS. 1 through 4, the present invention may include a geriatric undergarment 10 dimensioned and adapted to be worn about the waist of a human wearer. The geriatric undergarment 10 is movable between an open condition and a closed condition through removably fasteners, such as adhesives, along a waistband portion thereof.

The geriatric undergarment 10 may be incorporated into a conventional diaper. The body of the geriatric undergarment 10 may be made of an impact resistant foam 1 material which in turn will be coated by pressure absorbent material 2 (for example, polyethylene film, cellulose, elastane, gel, adhesive tapes, compressed airbags, hydro-sensitive or the like) located along an inner layer of the geriatric undergarment 10 so as to be disposed along diaper pressure regions, including the lateral (right and left), sacral, or posterior and trochanteric and/or posterior dorsal regions. This structure may be secured to the inside of a conventional diaper by adhesive material 3, and still function to dissipate the pressure exerted on said diaper pressure regions along the skin of the wearer.

The geriatric undergarment 10 provides structural protection against pressure ulcers by disposing the pressure absorbent material 2 to be located adjacent the right and left lateral regions, in addition to the posterior part, are coated with a layer of pressure absorbent material 2. The coated pressure absorbent material 2 may be shaped in pyramidal or tapered, or in the form of grooves which are arranged on the inner surface of the geriatric undergarment 10. When the coated pressure absorbent material 2 contacts or engages the skin of the wearer along said diaper pressure regions, the coated pressure absorbent material 2 functions to dissipate the pressure at these points of greatest contact.

Size and capacity indications may be printed on the outside of the diapers, as well as directions from where each part of the structured coated pressure absorbent material 2 should be secured within the disposable geriatric undergarment 10, to clarify the mode of use better.

Also, the geriatric undergarment 10 may be provided so that the disposition of the pressure absorbent material 2 prevents rashes for adults or infants.

It should be understood, of course, that the foregoing relates to exemplary embodiments of the invention and that modifications may be made without departing from the spirit and scope of the invention as set forth in the following claims.

What is claimed is:

1. A geriatric undergarment, comprising:
a body dimensioned and adapted to be worn about the waist of a human wearer; and
one or more pressure absorbent attachment points disposed along an inner surface of the body so that each pressure absorbent material is located adjacent to one or more diaper pressure regions of the wearer.

2. The geriatric undergarment of claim 1, wherein the one or more diaper pressure regions of the wearer comprise a lateral, sacral, posterior, trochanteric or a posterior dorsal region.

3. The geriatric undergarment of claim 1, wherein the one or more diaper pressure regions of the wearer comprise a lateral, a sacral, a posterior, a trochanteric and a posterior dorsal region.

4. The geriatric undergarment of claim 1, wherein one or more of the pressure absorbent attachment points are shaped in pyramidal form.

5. The geriatric undergarment of claim 1, wherein one or more of the pressure absorbent attachment points are shaped in tapered form.

6. The geriatric undergarment of claim 1, wherein the body comprises impact resistant foam.

7. The geriatric undergarment of claim 1, wherein the one or more pressure absorbent attachment points are adhered to the inner surface by adhesive.

8. The geriatric undergarment of claim 7, wherein the one or more of the pressure absorbent attachment points comprise polyethylene film, cellulose, elastane or gel.

9. A geriatric undergarment, comprising:
a body of impact resistant foam dimensioned and adapted to be worn about the waist of a human wearer; and
one or more pressure absorbent attachment points adhered via adhesive along an inner surface of the body so that each pressure absorbent material is located adjacent to the one or more diaper pressure regions of the wearer comprising a lateral, sacral, posterior, trochanteric and a posterior dorsal region, wherein the one or more of the pressure absorbent attachment points are shaped in pyramidal or a tapered form.

10. The geriatric undergarment of claim 9, wherein the one or more of the pressure absorbent attachment points comprise polyethylene film, cellulose, elastane or gel.
